(19) United States
(12) Patent Application Publication
(10) Pub. No.: US 2003/0106105 A1
(43) Pub. Date: Jun. 5, 2003

(54) Promoter expressed specifically in the cells of plant roots, recombinant vectors and host cells comprising same and transgenic plants obtained

(75) Inventors: Beate Hoffmann, Magny les Hameaux (FR); Pascale Mollier, Gif sur Yvette (FR); Georges Pelletier, Bures sur Yvette (FR)

Correspondence Address: Mark B. Wilson, Fulbright & Jaworski L.L.P., Suite 2400, 600 Congress Avenue, Austin, TX, 78701, US

(73) Assignee: Institut Natioanl de la Recherche Agronomique (INRA), Paris Cedex 07 (FR)

(21) Appl. No.: 10/027,880

(22) Filed: Dec. 21, 2001

Related U.S. Application Data
(63) Continuation of application No. PCT/FR00/01768, filed on Jun. 23, 2000.

(30) Foreign Application Priority Data
Jun. 25, 1999 (FR) 99/08185

(51) Int. Cl. A01H 5/00; C07H 21/04; C12N 9/24; C12N 15/82; C12P 21/02; C12N 5/04

(52) U.S. Cl. 800/287; 536/23.2; 435/69.1; 435/200; 435/320.1; 435/419

(57) ABSTRACT

The invention concerns a novel plant promoter capable of directing the expression of a nucleotide sequence of interest in the cells of a plant root, and recombinant vectors containing such a promoter, preferably associated with a nucleotide sequence whereof the expression is desired in constitutive cells of plant roots.

The present invention relates to a novel plant promoter capable of directing the expression of a nucleotide sequence of interest in the cells of the root of a plant as well as recombinant vectors containing such a promoter, preferably associated with a nucleotide sequence whose expression is desired in the cells constituting plant roots.

In recent years the industrial applications made possible by the transformations of plants with the aid of genetic engineering have been increasing.

Many genes of prokaryotic or eukaryotic (of plants or animals) origin coding specifically for proteins conferring novel agronomic properties have been isolated and transferred to plants by genetic engineering.

In very many cases the genes which were introduced into plants constitute chimeric sequences, associating regulatory elements of different origins.

Thus, the gene coding for a protein of interest is often placed under the control of a strong constitutive promoter allowing the said protein to be expressed throughout the plant.

As an example, the promoter of the 35S transcript of the cauliflower mosaic virus (35S CaMV) has been widely used in constructions of chimeric genes for the expression of proteins of interest in plants.

Henceforth, for a large number of applications, it is not necessary for the expression of the protein of interest conferring the desired agronomic property to be disseminated throughout all of the organs and/or cell types of the transformed plant.

Very early, the search for a more specific expression of the gene of interest was undertaken and led, for example, to the identification of tissue- or organ-specific promoters.

In particular, a promoter directing the expression of a polynucleotide of interest in a manner both strong and targeted in the root would allow many applications that may be classed as follows:

(i) defence against the pathogens at the site of entry into the root, such as bacteria, fungi, nematodes or insects

(ii) resistance to stress (cold, hydric stress, salt stress);

(iii) improvement of quality (example: increase the sucrose content in sugar beet);

(iv) nutrition (example: express a transporter gene for nitrates).

As already indicated above, the promoters described in the state of the art do not allow the expression of a polynucleotide of interest in all of the cellular layers of the root including all of the strata.

For example, the arsk1 gene of A. thaliana (Hwang et al., 1995 is specifically expressed in the root, but its expression is limited to the external layers of the root (epidermis, endoderm, cortex), i.e. the cells implicated in water absorption. The expression is very weak in the vascular system. The expression profile of this gene suggests a role in hydric stress. As a result, the expression of this gene is inducible by hydric stress (exposure of roots to the air or treatment of the roots by ABA or NaCl) and diminishes considerably when the roots are rehydrated.

Another illustration is the scarecrow mutant of A. thaliana (Malamy et al. 1997) which is affected in the radial organization of the root: the layers of the endoderm and cortex do not assume a separate identity and remain fused in a mutant layer possessing characteristics of the endoderm and the cortex. The scarecrow gene affected by the mutation is expressed in the endoderm, the initial cells of the endoderm and sometimes in the quiescent centre of the root.

Furthermore, the promoters described in the state of the art, on the one hand, do not allow a high level of expression of the polynucleotide of interest and, on the other, are not active throughout the development of the plant.

The need for a strong plant promoter specific for the roots and active irrespective of the stage of development of the plant is henceforth made good according to the present invention.

The applicant has thus isolated from the plant genome of Arabidopsis thaliana a novel promoter capable of directing the expression of a polynucleotide of interest specifically in the roots of a plant, said promoter ensuring a high level of expression of the polynucleotide of interest simultaneously in the epidermis, the cortex, the vessel or the endoderm as well as in all of the strata of the root, and does so throughout all the stages of plant development.

Thus, the present invention relates to an isolated nucleic acid characterized in that it comprises a polynucleotide coding for a plant promoter capable of directing the expression of a nucleotide sequence of interest in the cells of the root of a plant throughout the entire development of this latter or to a nucleic acid with a complementary sequence.

Preferably, a nucleic acid according to the invention is available in an isolated or purified form.

The term “isolated” in the sense of the present invention designates a biological material which has been removed from its original environment (the environment in which it is situated naturally). For example, a polynucleotide present in the natural state in a plant or an animal has not been isolated. The same polynucleotide separated from the adjacent nucleic acids within which it is naturally inserted in the genome of the plant or animal is isolated.

Such a polynucleotide may be included in a vector and/or such a polynucleotide may be included in a composition and nonetheless remain in the isolated state as a result of the fact that the vector or the composition does not constitute its natural environment.

The term “purified” does not require that the material is present in an absolutely pure form, free from the presence of other substances. It is rather a relative definition.

A polynucleotide is in the purified state after purification of the starting material or the natural material by at least one order of magnitude, preferably 2 or 3 and most preferred 4 or 5 orders of magnitude.

For the purposes of the present description, the expression “nucleotide sequence” may be employed to designate indiscriminately a polynucleotide or a nucleic acid. The expression “nucleotide sequence” includes the genetic material itself and is therefore not limited to information concerning its sequence.

The invention also relates to a nucleic acid characterized in that it comprises all or part of a polynucleotide possessing at least an 80% nucleotide identity with the nucleotide sequence SEQ ID No. 1, or a nucleic acid with a complementary sequence.

The “percentage nucleotide identity” between two sequences in the sense of the present invention may be defined by comparing two sequences optimally aligned through a window of comparison. The part of the nucleotide sequence in the window of comparison may thus include additions or deletions (for example “gaps”) with respect to the reference sequence (which does not include these additions or these deletions) so as to obtain an optimal alignment of the two sequences.

The percentage is calculated by determining the number of positions at which an identical nucleotide base is observed for the two sequences compared, then by dividing the number of positions at which there is identity of the two bases by the total number of positions in the window of comparison, then by multiplying the result by 100 in order to obtain the percentage sequence identity.

The optimal alignment of the sequences for the comparison may be achieved by computer with the aid of known algorithms (for example, FASTA software of the WISCONSIN GENETICS SOFTWARE PACKAGE company, GENETICS COMPUTER GROUP (GCG), 575 Science Doctor, Madison, Wis).

As an illustration, it will be possible to determine the percentage sequence identity with the aid of the previously mentioned FASTA software, by using exclusively the default parameters.

Thus, the nucleotide differences that a nucleic acid according to the invention may comprise in comparison with the nucleotide sequence SEQ ID No. 1 may or may not result in substitutions, deletions or additions of one or several consecutive nucleotides.

Also included in the invention are nucleic acids comprising all or part of a polynucleotide possessing at least 85%, 90%, 95%, 98%, 99%, 99.5% or even 99.8% of nucleotide identity with the nucleotide sequence SEQ ID No. 1, or a nucleic acid with a complementary sequence.

According to another feature, the invention also relates to a nucleic acid characterized in that it comprises all or part of a polynucleotide hybridizing under hybridization conditions of high stringency with the nucleotide sequence SEQ ID No. 1, or a nucleic acid with a complementary sequence.

By “part” of a polynucleotide promoter according to the invention is meant a nucleotide sequence of a length of bases shorter than that of the sequence SEQ ID No. 1 which conserves the capacity to direct the expression of a nucleotide sequence of interest in the cells of the root of a plant.

The biological activity of a part of a polynucleotide promoter according to the invention can be easily verified by the specialist skilled in the art, in particular with the aid of vector constructions and procedures for plant transformations with the latter, such as are described in the examples.

By “part” of a promoter according to the invention is meant in particular the following candidate sequences:

the polynucleotide extending from the nucleotide in position 1 to the nucleotide in position 2400 of sequence SEQ ID No.3;

the polynucleotide extending from the nucleotide in position 493 to the nucleotide in position 2400 of sequence SEQ ID No.3;

the polynucleotide extending from the nucleotide in position 1076 to the nucleotide in position 2400 of sequence SEQ ID No.3;

the polynucleotide extending from the nucleotide in position 1976 to the nucleotide in position 2400 of sequence SEQ ID No.3; an

the polynucleotide extending from the nucleotide in position 2040 to the nucleotide in position 2400 of sequence SEQ ID No.3.

As an illustration, a part of a polynucleotide promoter according to the invention can be obtained by enzymatic cleavage of a nucleic acid such as described above, in particular a nucleic acid of sequence SEQ ID No.1 with the aid of restriction endonucleases.

A “part” of a polynucleotide promoter according to the invention can also be obtained for example by deletion of one or several nucleotides of the polynucleotide sequence SEQ ID No. 1 with the aid of the exonuclease III technique described in the examples. A polynucleotide part of the plant promoter according to the invention advantageously has a nucleotide length ranging from 200, 250, 300, 400, 500, 750, 1000, 1200, 1500 or 2000 nucleotides (or base pairs if it exists in the double-stranded form).

For this purpose, the specialist skilled in the art can use the restriction map of the nucleotide sequence SEQ ID No.1, shown in FIG. 1.

For the use of restriction enzymes for the purposes of obtaining polynucleotide fragments corresponding to a part of a polynucleotide promoter according to the invention, the specialist skilled in the art will advantageously be able to refer to the monograph by Sambrook et al. (1989, Molecular Cloning: A Laboratory Manual. 2 ed. Cold Spring Harbor Laboratory, Cold Spring Harbor, N.Y.).

A part of a polynucleotide promoter according to the invention can also be prepared by specific amplification of the fragment of interest with the aid of a primer couple flanking the sequence of interest from the 5′ side and the 3′ side, respectively, for example with the aid of the PCR method such as described in particular in the American patents U.S. Pat. Nos. 4,683,195, 4,683,202 and 4,965,188.

By “hybridization conditions of high stringency” in the sense of the present invention is meant the following hybridization conditions:

prehybridization of the filters for 8 hours at 65° C. in a buffer composed of 6×SSC, 50 mM Tris-HCl (pH 7.5), 1 mM EDTA, 0.02% PVP, 0.02% Ficoll, 0.02% BSA and 500 μg per ml of denatured salmon sperm DNA;

hybridization of the filters for 48 hours at 65° C. in the presence 1×SSC buffer corresponding to 0.15 M NaCl and 0.05 M sodium citrate;

three washes of the filters in a solution containing 2×SSC and 0.1% SDS at 68° C. for 15 minutes.

The hybridization conditions described above are adapted to the hybridization under highly stringent conditions of a nucleic acid molecule 20 nucleotides long.

It is obvious that the hybridization conditions described above must be adapted as a function of the length of the nucleic acid whose hybridization is desired according to techniques well-known to the specialist skilled in the art.

Suitable hybridization conditions may be adapted in accordance with the teaching contained in the monograph of Hames and Higgins (1985, Nucleic Acid Hybridization: A practical approach, Hames and Higgins Ed., IRL Press, Oxford) or also in the monograph of Sambrook et al. (1989) previously mentioned.

The invention also relates to a nucleic acid containing a polynucleotide promoter such as defined above, characterized in that it comprises in addition a nucleotide sequence of interest functionally associated with the plant promoter and whose expression is desired in the cells of the root of a plant.

A nucleic acid fulfilling such a definition is for example the nucleic acid of the nucleotide sequence SEQ ID No. 2 comprising the sequence of the gus gene placed under the control of the promoter of nucleotide sequence SEQ ID No. 1.

Advantageously, such a nucleic acid will comprise a nucleotide sequence of interest selected from the gene coding sequences interacting with parasites or pathogens such as nematodes or fungi such as for example the sequences coding for glucanase, said nucleotide sequence of interest being placed under the control of a polynucleotide promoter according to the invention.

It may also relate to endochitinase sequences such as those described in the European patent No. EP 493,581 or also gene sequences acting on the sugar content of the plant.

As an example, the coding sequences of genes of interest ensuring the protection of a plant against other conditions of stress can advantageously be placed under the control of a polynucleotide promoter according to the invention.

Water or salt stress:

arsk 1 gene (Hwang, I et al.; 1995);

CDNA pA9 (Winicov, I., Deutsch S. E. 1994);

CDNA Alfin 1 (Bastola, D R et al. 1998).

Other coding sequences might be used under the control of the promoter according to the invention to act on the sucrose content of the sugar beet: the BvSPS1 gene (Hesse H. et al., 1995), or to overexpress a gene already expressed physiologically like the nitrate transporter genes NRT1 or NRT2 (Crawford, N. M. et al., 1998; Leah, R. et al., 1991).

The invention also relates to nucleotide fragments comprising 10 to 2000 consecutive nucleotides of a nucleic acid according to the invention, in particular of a nucleic acid possessing at least 80% nucleotide identity with the sequence SEQ ID No. 1 or also a nucleic acid hybridizing under hybridization conditions of high stringency with the nucleotide sequence SEQ ID No. 1, or a nucleic acid with a complementary sequence.

Preferably, such fragments will have lengths of 10, 12, 15, 18 or 20 to 25, 35, 40, 50, 70, 80, 100, 200, 500, 1000, 1500 or 2000 consecutive nucleotides of a polynucleotide promoter according to the invention or consist of fragments 12, 15, 18, 20, 25, 35, 40, 50, 70, 80, 100, 200, 500, 1000, 1500 or 2000 consecutive nucleotides long of a polynucleotide promoter according to the invention.

Such nucleotide fragments can advantageously be used as probes or nucleotide primers for the purposes of detection or amplification of all or part of a sequence with promoter activity specific for the roots of plants according to the invention.

According to another feature, the invention relates to a recombinant cloning and/or expression vector comprising a polynucleotide promoter according to the invention. Such a recombinant vector advantageously comprises a nucleotide sequence of interest placed under the control of said plant promoter.

Vectors which can be used for the purposes of the present invention are in particular the following:

vector pBIN19 (Bevan et al., 1984, Nucleic Acids Research, vol. 12: 8711-8721, sold by the CLONTECH company, Palo Alto, Calif., USA);

vector 101 (Jefferson, 1987, Plant Molecular Biology Reporter, vol.5: 387-405, sold by the CLONTECH company);

vector pBI221 (Jefferson, 1987, Plant Molecular Biology Reporter, vol.5: 387-405, sold by the CLONTECH company);

vector pBI121 (Jefferson, 1987, Plant Molecular Biology Reporter, vol.5: 387-405, sold by the CLONTECH company);

vector pEGFP (Cormack, B. P. et al. 1996; Yang T. T. et al., 1996), sold by the CLONTECH company.

Vector pc-gus shown in FIG. 10.

A preferred recombinant vector according to the invention is, for example, the recombinant vector contained in the E. coli strain deposited with the National Collection of Cultures of Micro-organisms (NCCM) on May 25, 1999 under the access No. I-2218.

The invention also relates to a recombinant host cell, characterized in that it contains a nucleic acid with plant promoter activity specific for plant roots according to the invention, optionally associated with a polynucleotide of interest placed under the control of this latter, or a recombinant vector such as defined above.

The preferred recombinant host cells according to the invention may be indiscriminately of bacterial or plant origin.

Thus, use may be made in particular of bacterial cells of different E. coli strains or also of Agrobacterium tumefaciens.

They may also be plant cells transformed by a vector in conformity with the invention, such as cells of Arabidopsis thaliana, colza, tobacco or also maize.

A preferred recombinant host cell according to the invention is the cell of the E. coli strain deposited with NCCM on May 25, 1999 under the access No. I-2218.

The invention also relates to a recombinant plant multicellular organism characterized in that it comprises recombinant host cells such as defined above.

The invention relates in particular to a transgenic plant comprising in a form integrated in its genome a nucleic acid according to the invention in particular a nucleic acid comprising a polynucleotide promoter in conformity with the invention and a nucleotide sequence of interest placed under the control of this latter.

A transgenic plant according to the invention may be in particular colza, tobacco, maize or also Arabidopsis thaliana.

The transgenic plants such as those defined above thus have the property of expressing a nucleotide sequence of interest specifically at the level of the different cell types of the root (from the exterior towards the interior: epiderm, cortex, endoderm, pericycle, vessel) at all stages of development of the plant.

The invention also relates to a procedure for obtaining a transgenic plant specifically expressing a nucleotide sequence of interest in the cells of the root at all stages of development of said plant, characterized in that it comprises the following steps:

a) production of a plant recombinant host cell conforming to the invention;

b) regeneration of an entire plant starting from the recombinant host cell obtained in step a);

c) selection of the plants obtained in step b) which have integrated the nucleotide sequence of interest placed under the control of the plant polynucleotide promoter according to the invention.

The invention also relates to a procedure for obtaining a transgenic plant characterized in that it comprises the following steps:

a) production of a recombinant host cell of Agrobacterium tumefaciens containing a nucleotide sequence of interest placed under the control of the plant polynucleotide promoter according to the invention;

b) transformation of the plant of interest by infection with the recombinant host cell of Agrobacterium tumefaciens obtained in step a);

c) selection of the plants obtained which have integrated the nucleotide sequence of interest placed under the control of the plant polynucleotide promoter according to the invention.

The invention also relates to a procedure for obtaining a transgenic plant characterized in that it comprises the following steps:

a) transfection of a plant cell with a nucleic acid or a recombinant vector containing a nucleotide sequence of interest placed under the control of the polynucleotide promoter according to the invention;

b) regeneration of an entire plant starting from the recombinant host cell obtained in step a);

c) selection of the plants obtained which have integrated the nucleotide sequence of interest placed under the control of the plant polynucleotide promoter according to the invention.

Any one of the procedures for obtaining a transgenic pplant described above may also comprise the following additional steps:

d) a cross between two transgenic plants such as those obtained in step c);

e) selection of the plants homozygous for the transgene.

According to another alternative, any one of the above procedures may in addition comprise the following steps:

d) a cross of a transgenic plant obtained in step c) by any one of these procedures with a plant of the same species:

e) selection of the plants derived from the cross in step d) which have conserved the transgene.

The invention also relates to a transgenic plant such as obtained according to any one of the above procedures.

Preferably, a transgenic plant according to the invention has not only integrated into its genome a transgene comprising a nucleotide sequence of interest placed under the control of the plant polynucleotide promoter presently described but expresses said nucleotide sequence of interest predominantly or exclusively in the constituent cells of the root.

Finally, the invention also relates to a plant seed, the constituent cells of which contain in their genome a nucleic acid according to the invention.

In particular it is a seed of Arabidopsis thaliana, colza, tobacco or maize which has incorporated a nucleic acid according to the invention.

The invention will in addition be illustrated by the Figures and the following examples, without in any way being limited by them.

FIG. 1 presents a restriction map of the nucleotide sequence SEQ ID No. 1.

The following motifs were identified in this sequence: two TGACG motifs corresponding to the binding site of the root-specific factor Asfl in the 35S promoter of the CaMV (position 1000-1004 and 1866-1870), two motifs close, to within one nucleotide, to enhancer sequences of the same 35S promoter (position 28-35: CTGAAAG instead of GTGAAAG and position 882-889: GTGCTTTG instead of GTGGTTTG) and 3G-box ACGT (positions 285-288, 604-607, 1107-1110). Moreover, this sequence contains 21 TATA motifs and 9 CAAT motifs.

The functional importance of these motifs can be evaluated by the method using exonuclease III, according to Ausubel et al. (1989). This method makes it possible to obtain promoter fragments of decreasing size which will be cloned upstream from the gus gene in a vector permitting the transformation of Arabidopsis.

FIG. 2 illustrates construction 1 which was used for the isolation of the promoter according to the invention, in the absence (FIG. 2a) or in the presence (FIG. 2b) of the insert.

The 4.27 kb insert is cloned starting from the “kanamycin rescue” vector (FIG. 7) in the T-DNA of the pBin19 vector by means of a double EcoRI-XbaI digestion. This insert contains 2.14 kb of genomic sequence of the clone Ir1 (SEQ ID No.3 nt 136-2284) and 2.13 kb of the T-DNA of pGKB5: gus coding sequence and nos polyadenylation signal (FIG. 8—nt 632-2762).

LB: left border of the pBin19 T-DNA.

LacZ: lacZ region of the phage M13mp19.

NPTII: fragment containing the nos promoter, the neomycin resistance gene and the nos polyadenylation site.

RB: right border of the pBin19 T-DNA.

kan: fragment containing the origin of replication RK2 of the plasmid pRK252 and the kan gene for kanamycin resistance of Streptococcus.

FIG. 3 illustrates the GUS expression of the Arabidopsis (ecotype WS) transformant during development.

a—7 days after germination.

b—14 days after germination

c—24 days after germination

d—detail of a root

FIG. 4 illustrates a transverse section through the root of the transformant after revelation of GUS activity.

FIG. 5 represents an autoradiography of a Northern blot hybridized with a GUS probe.

6 μg of RNA were deposited in each well.

Wells No. 1-3-5: RNA of the aerial parts of the homozygous transformant No. 1, No. 13 and of untransformed WS plant, respectively.

Wells No. 2-4-6: RNA of the roots of the same plants

FIG. 6 illustrates the quantitative analysis of the GUS expression of Arabidopsis transformants obtained with construction 1 during development. It represents the comparison of the GUS activity in the roots and the aerial part of the initial transformant (a) and of the characteristic individual transformants 6-1 and 2b (b-c) during development.

The gus activity is expressed in fluorescence units per minute and per:

1 μg of proteins (roots)

20 μg of proteins (leaves)

2.72 fluorescence units correspond to 1 pmol of the product mu, which is the product of enzymatic catalysis of the substrate mug (4-methyl-β-D glucuronide) by GUS.

FIG. 7 illustrates the vector obtained following “kanamycin rescue”. The “kanamycin rescue” technique uses the vector P38 (a), which carries the beginning of the Nptil gene for kanamycin resistance up to the PstI site, downstream from a promoter IS50. After PstI digestion of the vector P38 and the DNA of the transformant, ligation of the two and selection on kanamycin, the vector shown in b) is obtained. The insert 1 is the PstI fragment obtained starting from the genomic DNA of the transformant: it contains the promoter region (SEQ ID No.1, nt 1 to 2149) joined to the T-DNA fragment delimited by the RB side of the insertion site and the PstI site situated in the kanamycin gene (FIG. 8, nt 632 to 4279). The kanamycin resistance gene is thus reconstituted and the recombinant vector is selected on kanamycin.

FIG. 8 presents a schematic representation of the T-DNA of pGKB5 used to create the collection of Versailles transformants.

FIG. 9 illustrates the T-DNA sequence of pGKB5, also entered under the reference sequence SEQ ID No. 5.

RB border of 24 bp: 574-596,

gus gene: gus sequence without promoter: 638-2504 (ATG: 638-640, stop codon: 2444-2446), gus polyadenylation site: 3′ nos: 2505-2793, EcoRI site: MTT/C: 2759-2763.

KanR gene: nos promoter: 4752-4480, KanaR sequence: 4479-3490 (ATG: 4466-4464, stop codon: 3665-3663, PstI site: CTGCA/G: 4275-4280), ocs 3′ site: 3489-2794.

PhosphinothricinR gene (bastaR): 35S promoter: 4767-5890, phosphinotricinR sequence: 5890-6503 (ATG: 5930-5932, stop codon: 6480-6482), g7 3′ site: 6504-6789.

LB border of 24 bp: 6962-6986

FIG. 10 represents a detailed map of the vector pC-gus used in Example 5.

### EXAMPLES

### MATERIALS AND METHODS

I—Transformation

(Bechtold N;, Ellis J., Pelletier G., 1993. Agrobacterium mediated gene transfer by infiltration of adult Arabidopsis thaliana plants. C. R. Acad. Sci. Paris 316: 1194-1199).

6 mg of seeds (i.e. about 300 seeds) of Arabidopsis thatiana of ecotype Wassilevskija were sown in 40×30 cm trays of compost. The trays were left to germinate for 64 h at 4° C., then placed in the greenhouse (photoperiod: 16 h of daylight, temperature: 15° C. at night/minimum of 25° C. during the day) and sprinkled with the standard nutritive solution of Coïc and Lessaint (Coïc, Y., Lessaint, C. 1971. Comment assurer une bonne nutrition en eau et ions minéraux en horticulture. Hortic. Fr.8: 11-14).

Agrobacterium MP5-1 is grown in LB medium (Luria-Bertani, Sambrook, J., Fritsch, E. F., Maniatis, T. 1989. Molecular Cloning: A Laboratory Manual. Cold Spring Harbor, N.Y.) with 50 mg/ml of rifampicin, 100 mg/l of gentamycin and 200 mg/l of kanamycin, 14 h at 28° C. (until A600=0.8). After centrifugation, the bacterial pellet is resuspended in one third of the initial volume of the infiltration culture medium (IM) (IM=macro and micro nutrients of Murashige and Skoog, containing 10μg/l of 6-benzylaminopurine and 5% sucrose (Murashige, T., Skoog, F. 1962. A revised medium for rapid growth and bioassays with tobacco tissue cultures. Physiol Plant 15: 473-497). Batches of 100 to 500 well-developed 3 to 4 weeks old plants were removed from the soil, rinsed with water and immersed in 21 of IM medium containing Agrobacterium in a vacuum bell jar of 101 capacity. The plants are maintained under vacuum (10⁴ Pa) for 20 min. Latex gloves were used throughout for handling the treated plants until they were harvested. The treated plants were planted in new compost, 54 plants per tray, then incubated for 2 days under plastic in order to prevent any dehydration and to facilitate the development of their root system. Four to six weeks after plantation, the T1 generation was harvested as a mixture. The plants were selected on sand irrigated with water containing the herbicide Basta (5-10 mg/ml phosphinothricin). Two months later the T2 seeds were harvested individually and stored for subsequent analyses.

II—“Kanamycin Rescue”

(Bouchez D., Vittorioso P., Courtial B., Camilleri C., 1996. Kanamycin Rescue: A simple technique for the recovery of T-DNA flanking sequences. Plant Mol. Biol. Rep. 14: 115-123).

Extraction of Genomic DNA

Leaves (0.5 to 0.75 g) are frozen rapidly in liquid nitrogen, ground in the presence of polyclar™ to a fine powder with a pestle and mortar and the powder is transferred with the liquid nitrogen into an “Oak Ridge” tube to which are added 15 ml of extraction buffer (100 mM Tris, 50 mM EDTA, 1500 mM NaC, 10 mM β-mercaptoethanol, pH 8). After addition of 1 ml of 20% SDS, the tubes are incubated at 65° C. for 10 min with shaking every 3 to 4 min. 5 ml of potassium acetate (5M) are added and incubated at 0° C. for at least 20 min. After centrifugation at 25,000 g (13000 rpm) for 20 min, the supernatant is filtered through a Miracloth filter (Calbiochem) into a 30 ml tube containing 10 ml of isopropanol and incubated at −20° C. for 30 min. After centrifugation at 20000 g (10,000 rpm) for 15 min, the DNA pellet is dried by inverting the tube on absorbent paper for 10 min. The DNA is taken up in 0.7 ml of 50/10 TE (50 mM Tris, 10 mM EDTA, pH 8 to which are added 5 μl of RNAse (5mg/ml) and incubated at 37° C. for 10 min. The DNA is extracted with an equal volume of 1/1 phenol/chloroform and precipitated by isopropanol (1 volume)/3M NaOAc (1/10 volume). The DNA pellet is dried and taken up in 10 μl of 10/1 TE (10 mM Tris, 1 mM EDTA, pH 8).

Cloning

First Digestion.

0.5 μg of Arabidopsis genomic DNA are digested with PstI (BRL Life Technologies, 95613, Cergy-Pontoise), precipitated with ethanol (2.5 volumes)/3M NaOAc (1/10 volume) and resuspended in water. 2.5 μg of the vector pResc38 are digested with PstI, dephosphorylated with calf intestine alkaline phosphatase (BRL), extracted with one volume of phenol-chloroform (1/1), precipitated with ethanol/NaOAc and resuspended in water.

First Ligation.

0.5 μg of genomic DNA digested with PstI and 2.5 μg of pResc38 digested with PstI and dephosphorylated are ligated in 100 μl total volume with 5 units of T4 DNA ligase (BRL), overnight at 12° C.

Second Digestion.

The preceding ligation mixture is precipitated with ethanol (2.5 volumes)/8M NH₄OAc (½ volume), resuspended in water and completely digested with a second restriction enzyme: XbaI, in a total volume of 100 μl using 20 units of restriction enzyme. The mixture is precipitated with ethanol/NH₄OAc and suspended in water.

Second Ligation.

In order to circularise the DNA molecules, a second ligation is carried out on the product of the second digestion with a lower DNA concentration, in a total volume of 200 μl and using 5 units of T4 DNA ligase. The mixture is incubated overnight at 12° C., then precipitated with ethanol/NH₄OAc, rinsed twice with 70% ethanol (v/v), dried and taken up in 20 μl of water.

Transformation

Electroporation is carried out using a Gene-Pulser (Bio-Rad Laboratories, Richmond, Calif.) type of apparatus with a voltage of 1.5 kV. The electromax DH10B electrocompetent cells (BRL) are rapidly thawed then placed on ice. 2 μl of the precipitated ligation product and 40 μl of competent cells are mixed in a cold electroporation cuvette (1 mm interelectrode diameter, Bio-Rad). After electroporation, 1 ml of cold SOC medium (Sambrook, J., Fritsch, E. F., Maniatis, T. 1989. Molecular Cloning: A Laboratory Manual. Cold Spring Harbor, N.Y.) is added immediately. The whole is decanted into a 13ml culture tube and incubated for 2h at 37° C. with shaking.

A culture volume of 250 μl is spread on LB-agar Petri dishes containing 100 mg/l of carbenicillin and 50 mg/l of kanamycin and incubated at 37° C. overnight.

III—Cloning in pBin19

The insert cloned in the “kanamycin rescue” vector P38resc undergoes an intermediate cloning in the vector Bluescript pBKS+(Stratagene, San Diego Calif. 92121) before being cloned in the binary vector pBin19 for the purpose of the transformation of the plants. These clonings are performed in a directional manner by double digestion EcoRI/XbaI.

About 250 ng of vector P38resc containing the insert and digested by EcoRI and XbaI are ligated with about 100 ng of the non-phosphorylated vector KS+ digested by the same enzymes in 40 μl final volume with 10 U of T4 DNA ligase (BRL). After incubation overnight at 12° C., the ligation mixture is precipitated with ethanol/NH₄OAc, taken up in 10 μl of water and used to electroporate NM522 bacteria (BRL), made electrocompetent according to the procedure described by Sambrook et al. (1989). The white positive colonies are selected on an LB-agar medium containing 40 mg/l of XgaI, 8 mg/l of IPTG (Genaxis Biotechnology, 78180 Montigny le Bretonneux) and 100 mg/l of carbenicillin.

III—Cloning in pBin19

The insert contained in pBKS+, after digestion with EcoRI and XbaI, is purified by electroelution from a 1% agarose gel according to the procedure of Sambrook et al. (1989). For cloning, 100 ng of the 4.3 kb insert and 100 ng of vector pBin19 (12 kb) previously digested with EcoRI and XbaI (i.e. an insert/vector molar ratio of 3/1) are mixed in 40 μl total volume with 10 μl of ligase (BRL) and ligated overnight at 12° C. After precipitation with ethanol/NaOAc, the ligation product is taken up in 10 μl of water and used to carry out the electroporation of the NM522 bacteria. The positive colonies are selected on Petri dishes with an LB-agar medium containing XgaI and IPTG as above and 50 mg/l of kanamycin.

IV—Method Using Exonuclease III

(Current Protocols in Molecular Biology, editors: F. M. Ausubel, R. Brent, R. E. Kingston, D. D. Moore, J. G. Seidman, J. A. Smith, K. Struhl; published by Wiley Interscience).

The 4.3 kb DNA fragment of interest was recloned in a pBluescript II KS+ vector at the EcoRI site of the polylinker. The plasmid of this clone was then isolated and purified by the “Qiagen-Midi-Preparation Tip 100” method (Qiagen) starting from 30 ml of a culture.

In order to be able to sequence in both directions, 5 μg plasmid were doubly digested by XhoI/KpnI,on the one hand, and by SpeI/SacI, on the other, in a volume of 50 μl each time. (100 ng of linearised plasmid of each digestion were kept for a check on the agarose gel). The remainder of the linearised plasmid at each digestion was precipitated with 95% ethanol (3 volumes) and 3M NaOAc (⅕ volume) for one hour in an ice bath . After centrifugation for 20 minutes at 13000 rpm at +4° C., the digested plasmid from each digestion was rinsed with 70% ethanol, dried at the “speed-vac” for 5 minutes and taken up in 50 μl ExoIII buffer diluted to 1× (0.66M Tris/HCl pH=8.0, 66 mM MgCl2, 50 mM DTT, 500 μg/ml BSA; USB, United States Biochemicals).

In order to create the deletions on each side 25 μl (2.5 μg) of each digestion were preincubated at 37° C. for 2 minutes, 0.8 μl of ExoIII (100u/μl; USB), i.e. 150 units of ExoIII per picomole of 3′ ends were added and reincubated at 37° C. Every minute 3 μl (300 ng) of DNA were sampled and placed immediately in Dry Ice (total samples=8). Then 3μl of water were added to each sample, and the samples were incubated for 10 minutes at 70° C. in order to inactivate the enzyme ExoIII. All the samples were placed in ice. After addition of 15 μl of nuclease S1 buffer (300 mM Na acetate pH 4.6,10 mM Zn acetate, 50% v/v glycerol) and 4 μl (4 units) of nuclease S1 (Gibco BRL), these samples were incubated for 20 minutes at room temperature. The nuclease S1 reaction was stopped by adding 5 μl of “stop” buffer (0.3M Tris/HCl pH 8.0, 0.05 M EDTA) to each sample. 8 μl Aliquots were withdrawn for a check on agarose gel.

The remaining volume (22 μl) of each sample was incubated for 20 minutes at 37° C. after having added 2 units of Klenow fragment and 1 μl of 0.25 mM dNTPs.

Finally the deleted molecules were recircularised by adding 1 μl (1 unit) of T4 DNA ligase (USB), 3 μl of 10× buffer (660 mM Tris/HCl pH=7.6, 66 mM MgCl₂, 100 mM DTT, 660 μm ATP) and 2 μl of water to each sample. The ligations were performed in a total volume of 30 μl and incubated at 16° C. overnight.

Then one third of the volume (10 μl) of the products derived from each ligation was used to transform 100 μl of E. coli DH5α competent cells by the calcium chloride method (Current Protocols in Molecular Biology, editors: : F. M. Ausubel, R. Brent, R. E. Kingston, D. D. Moore, J. G. Seidman, J. A. Smith, K. Struhl; published by Wiley Interscience). The transformed E. coli DH5α cells were selected on LB-agar containing 100 mg/l carbenicillin, 40 mg/l of XgaI and 8 mg/l of IPTG.

V—Extraction of the Total RNAs

(Heim U., Weber H., Baumlein H., Wobus U., 1993. A sucrose synthase gene of Vicia faba L.: expression pattern in developing seeds in relation to starch synthesis and metabolic regulation. Planta 191: 3494-3501).

The frozen fresh tissues (plantlet˜2g and root˜1g) were crushed in liquid nitrogen by means of a pestle and mortar. Then 500 μl of extraction buffer (1 M Tris/HCl pH 7.4, 1% SDS, 5 mM EDTA) were added dropwise per 200 mg of tissue, followed by the same volume of phenol/chloroform/isoamyl alcohol while grinding was continued until a glossy powder was obtained. After thawing, each solution was transferred to a tube and centrifuged for 5 minutes at +4° C.

Each aqueous phase was re-extracted twice with the same volume of phenol/chloroform/isoamyl alcohol and precipitated with ethanol (3 volumes)/NaOAc (1/10 volume) for one hour at −80° C. After centrifugation for 30 minutes at +4° C. each pellet was dried briefly and dissolved in water+DEPC. A second centrifugation for 10 minutes at +4° C. was carried out and each supernatant was mixed with the same volume of 4M LiCl in order to precipitate the ribonucleic acids in ice at +4° C. overnight.

Each solution was then centrifuged for 15 minutes and the RNA pellets were washed twice with 2M LiCl and once with 70% ethanol. After drying at the “speed-vac”, each RNA pellet was dissolved in water+DEPC and the RNA concentration was checked by means of spectrophotometry.

VI—GUS Test

(Jefferson R. A., 1987. Assaying chimeric genes in plants: the gus gene fusion system. Plant Mol. Biol. Rep. 5: 387)

By Histochemistry

Two weeks after the germination of the Arabidopsis transformants, the GUS activity is tested using X-glucuronic acid (X-Glu, Biosynth G. Staad, Switzerland) as described by Jefferson et al., modified by the use of 100 mM KH₂PO₄, 0.4 mM of K₃Fe(CN)₆ and 0.4 mM K₄Fe(CN)₆ catalyst. No background noise was observed in the tissues of the non-transformed plants.

By Fluorimetry

The plant samples (roots and leaves) are ground in an Eppendorf™ tube with 200 μl of extraction buffer (50 mM NaPO₄, 10 mM dithiothreitol, 10 mM EDTA, pH7) and a pinch of Fontainebleau sand. After centrifugation twice for 10 min at 13,000 rpm at 4° C., the determination of GUS activity is made on the supernatant in a final volume of 150 μl containing the substrate MUG (umbelliferyl 4-methyl-β-D-glucuronide, Sigma) at a final concentration of 3 mM.

After incubation for 15 min. at 37° C., the GUS activity is measured using a Fluoroskan II apparatus (Labsystems, 91944 Le Ulis, France) with excitation and emission wavelengths of 365 nm and 455 nm, respectively.

The protein concentrations in the plant extracts are measured by using the Bradford reagent (Biorad).

The DNA concentrations are measured using the Hoechst reagent (Sigma). The reaction is performed in a final volume of 200 μl (Labarca-Paigen buffer: 50 mM NaPO₄, 2M NaCl, 2 mM EDTA, pH 7.5) containing the Hoechst reagent at 0.5 mg/ml.

### EXAMPLE 1

Isolation of a Nucleotide Sequence of About 2.2 kb by Promoter Trapping

A collection of Arabidopsis thaliana (ecotype WS) transformants was obtained according to the procedure described by Bechtold et al. (1993).

The plants were transformed by random insertion in their genome of transfer DNA (T-DNA) transmitted by the bacterium Agrobacterium tumefaciens.

This transfer DNA contains a gus gene without a promoter as described by Bouchez et al. (1993, C.R.A.S. Paris, volume 316: 1188-1193).

The method of transformation In planta was chosen and developed at the Station G6netique de Versailles de l'Institut National de la Recherche Agronomique according to the method described by Bechtold et al. (1993, C.R.A.S. Paris, volume 316: 1194-1199). This method makes it possible to rapidly obtain a large number of independent transformants comprising a limited number of insertions (1.5 insertions per transformant on average).

A histochemical screening of the expression of the GUS gene among the transformants according to the method described by Mollier et al. (1995, C.R.A.S. Paris, volume 318: 465-474) made it possible to isolate a transformant exhibiting a particular GUS activity:

very high expression specifically in the root throughout development as shown in the plates corresponding to FIG. 3a-c. The root is stained over its entire length except for the elongation zone (FIG. 3d).

expression in all of the cellular strata of the root (epidermis, cortex, endoderm, pericycle, conducting vessel) such as may be observed on the plate of FIG. 4.

This transformant was characterized further by means of the Southern blot procedure (Southern E.M., 1975).

A sequence of about 2.2 kb situated upstream from the right border of the insertion corresponding to the promoter was cloned by the “kanamycin rescue” procedure according to the technique described by Bouchez et al. (1996, Plant Mol. Biol. Rep. Vol.14: 115-123).

The “kanamycin rescue” vector is shown in FIG. 7.

### EXAMPLE 2

Search for the Complete Sequence of the Promoter According to the Invention.

The 2.2 kb DNA fragment was used as probe in order to search for the entire promoter in a genomic DNA library of Columbia ecotype Arabidopsis thaliana (J. T. Mulligan, Stanford Calif. 94305).

Two phages of about 15 kb were selected (clones Ir1 and Ir2). These two phage clones contained an insert corresponding to a 4,413 kb genomic fragment (SEQ ID No. 3) and containing the sequence of the probe. The insert of these two phages was sequenced completely by the exonuclese III method described by Ausubel et al. (Current Protocols in Molecular Biology, editors: F. M. Ausubel, R. Brent, R. E. Kingston, D. D. Moore, J. G. Seidman, J. A. Smith, K. Struhl; published by Wiley Interscience). It is the sequence SEQ ID No. 3. The start of the sequence corresponding to the T-DNA is localised starting from the nucleotide in position 2285 of the sequence SEQ ID No. 3.

The specific expression of the gus gene was detected by Northern blot experiments on total RNAs extracted from transformants homozygous for the insertion.

The results of a Northern blot experiment are shown in FIG. 5.

A transcript of about 2 kb is detected in the root RNAs and is not detectable in the RNAs of the aerial parts (FIG. 5).

The total RNAs were extracted from roots and aerial parts of the line transformed according to the method described by Heim et al. (1993, Planta, vol. 191: 3494-3501).

In order to detect a possible endogenous transcript corresponding to the promoter, Northern blot gels were carried out on total RNAs extracted from non-transformed plants and hybridized with the 4,413 kb genomic fragment (SEQ ID No. 3) which contains about 2.2 kb downstream from the promoter.

No transcript was detected with this probe.

In addition, two independent libraries of Arabidopsis thaliana cDNA (one library of roots cDNA and one library of whole plant cDNA) were screened with this same 4,413 kb probe.

Again the results were negative and no cDNA corresponding to the promoter was found.

Finally, no coding phase could be detected downstream from the promoter using the conventional prediction software. Software Net Plant Gene and Net Gene 2:

S. M. Hebsgaard et al. (1996)

Brunak S. et al. (1991 Software Genscan:

Burge, C et al. (1997);

Burge, C. B. (1998).

The 4,413 kb sequence (SEQ ID No. 3) is very rich in bases A and T (68% of A and T) and contains 67 ATG motifs, 20 CMT motifs, 38 TATA motifs, 9 TATMT motifs and 2 Cr boxes.

The results obtained indicate that no transcript is detectable downstream from the promoter studied. Hence it is a cryptic promoter.

### EXAMPLE 3

Detection of Promoter Activity

Promoter activity was demonstrated by carrying out a retransformation in planta of Arabidopsis thaliana (ecotype WS) by this 2.2 kb promoter placed upstream from the gus reporter gene.

For this experiment the following construction was carried out, which is shown in FIG. 2: a fragment of about 4.27 kb included between the XbaI and EcoRI sites of the “kanamycin rescue” vector (cf. FIG. 7) was cloned in the T-DNA of the pBin19 vector according to the procedure described by Bevan M (1984, Nucleic Acid Research vol.12: 8711-8721)

This 4.27 kb DNA fragment is included in the SEQ ID No. 4 sequence; this sequence also comprises a cloning polysite of the vector P38, as described below.

It comprises: the P38 cloning sites: XbaI, SpeI, BamHI, SmaI, PstI (nt 1 to 29), the promoter sequence SEQ ID No. 1 (nt 30 to 2178) and the sequence of the gus gene of the T-DNA of pGKB5 up to the EcoRI site (nt 2179-4309).

### EXAMPLE 4

Transformation of Arabidopsis thaliana Plants with the Construction Containing the Gus Gene Placed Under the Control of the Promoter.

Arabidopsis thaliana plants were transformed by means of Agrobacterium tumefaciens with the construction 1 described in FIG. 2 and nine individual transformants were studied for the expression of the gus gene, firstly by histochemistry.

The expression of the gus gene was also quantified by fluorimetric determination according to the procedure described by Jefferson (1987, Plant Mol. Biol. Rep. Volume 5: 387), modified by the use of 5 mM of substrate in the roots, on the one hand, and in the aerial parts (cotyledons, leaves, stems), on the other, and was performed at several stages of the development of the plants.

The activity of the gus gene of the nine transformants was compared to that of the initial transformant.

The results are shown in Table I below.

In the case of the initial transformant ACC6H in the homozygous state or ACC6T3 in segregation, the activity of the gus gene in the roots diminishes with the age of the plant whereas a low activity in the aerial parts becomes detectable at the end of development.

In the case of 6 of the 9 transformants studied (transformants 6i, 6h, 6-1, 6-2, 6-3 and 6a) the activity of the gus gene in the roots is even higher than that of the initial transformant (4 to 10 fold) and the activity in the leaves becomes more easily detectable.

However, the ratio: gus gene activity in the roots/gus gene activity in the leaf remains constant.

In the case of these transformants, the root specificity is hence unchanged with respect to the initial transformant. Solely the level of expression of the gus gene is higher overall.

For three of the transformants (6b, 2b and 6k) the ratio: gus gene activity in the roots/gus gene activity in the leaf is less than in the initial transformant; the expression of the gus gene is thus less specific for the roots in the case of these transformants.

The diminution of the GUS activity in the roots during development is illustrated in FIG. 6 for the initial transformant (a) and two characteristic transformants (b and c). For the initial transformant, the activity in the leaves is not detectable. In the case of the transformant 6-1, it is weakly detectable and the ratio: gus activity roots/leaf is the same as for the initial transformant. For the transformant 2b, on the other hand, the GUS activity in the leaves is higher and the root/leaf ratio is clearly diminished.

### EXAMPLE 5

Study of Deletions in the Promoter According to the Invention

Deletions in the promoter were obtained according to the exonuclease III method described in the materials and methods section (Section IV) in order to obtain functional fragments of the promoter.

Digestion of the Genomic Fragment by Means of the Exonuclease III

In the first place, the 4.3 kb genomic fragment (sequence SEQ ID No. 3) was cloned in a pBluescript KS+ vector at the EcoRI site, then subjected to partial digestions at 5′ by the exonuclease III.

Cloning of the Fragments Obtained in a Functional Expression Vector in the Plants

In order to test the promoter activity of the deleted fragments of the promoter, the fragments obtained after enzymatic digestion by means of the exonuclease III in the pBluescript KS+ vector were amplified, then cloned in a vector possessing the gus gene.

The fragments of the promoter are amplified by PCR with the aid of two primers bearing, respectively, enzymatic sites:

at the 5′ end of the promoter, the primer T7-HindIII located in the KS+ vector is used;

at the 3′ end of the promoter a primer chosen in the 4.3 kb genomic sequence and bearing a BamHI site is used:

The primers are the following:

a) primer T7-HindIII at 5′: GGC AAG CTT GTA ATA CGA CTC ACT ATA GGG C (SEQ ID No. 6) which possesses the sequence “A/AGCTT” recognized by the restriction endonuclease Hind III.

b) primer at 3′: CTA GGG ATC CAG CCA TTC CCT ATG C (SEQ ID No. 7) which possesses the sequence “GGATC/C” recognized by the restriction endonuclease BamHI. The sequence of this primer located at the 5′ end with respect to the BamHI site is complementary to the sequence extending from the nucleotide in position 2400 to the nucleotide at position 2386 of the sequence SEQ ID No. 3.

Protocol for Amplification by PCR

For each sample the following are mixed:

40 μl of water

5 μl of PCR buffer 10×

1 μl of 10 mM dNTP

1 μl of enzyme pfu-turbo DNA polymerase (at 2.5 u/μl, Stratagene)

1 μl of T7-HindIII primer (at 10 mM)

1 μl of 4.4-BamHI primer (at 10 mM)

1 μl of matrix DNA (10 ng of DNA of the chosen exonuclease clone)

PCR Reaction:

The actual amplification is carried out under the following conditions:

a) Denaturation step to obtain single-stranded DNA fragments at 94° C. for 4 minutes;

b) Thirty amplification cycles performed under the following conditions:

denaturation at 94° C. for 30 seconds;

hybridization of the primers at 50° C. for 45 seconds;

elongation of the primers at 72° C. for 3 minutes

c) Last elongation step performed at 72° C. for 10 minutes.

The promoter fragments thus amplified contain the HindIII site at 5′ and the BamHI site at 3′.

The amplified fragments are then cloned at the HindIII and BamHI sites, hence in an oriented manner, in the vector pC-gus, the detailed map of which is shown in FIG. 10. The cloning was carried out in conformity with the procedure described in the Materials and Methods section (section III) for the pBIN19 vector.

The fragments cloned upstream from the gus gene in the vector pC-gus are the following:

The fragment extending from the nucleotide at position 1 to the nucleotide at position 2400 of the sequence SEQ ID No. 3;

The fragment extending from the nucleotide at position 493 to the nucleotide at position 2400 of the sequence SEQ ID No. 3;

The fragment extending from the nucleotide at position 1076 to the nucleotide at position 2400 of the sequence SEQ ID No. 3;

The fragment extending from the nucleotide at position 1976 to the nucleotide at position 2400 of the sequence SEQ ID No. 3;

The fragment extending from the nucleotide at position 2040 to the nucleotide at position 2400 of the sequence SEQ ID No. 3;

Transformation of Agrobacterium tumefaciens cells with the recombinant vectors containing various fragments of the promoter according to the invention.

The pc-gus vectors containing the different inserts are then transferred to the Agrobacterium strain and Arabidopsis WS plants are transformed in conformity with the protocol described in Section I of the Materials and Methods section.

The seeds of the primary transformants are selected on a selective medium containing hygromycin (30 mg/l).

The descendants of 20 primary transformants by construction are sown on hygromycin medium in order to select the transformants possessing a single insertion locus of the Agrobacterium tumefaciens T-DNA. The homozygotes of these transformants are studied for the expression of the GUS protein in the roots and in the leaves, both qualitatively by histochemistry and quantitatively by fluorimetry, in conformity with the protocols described in Section VI of the Materials and Methods section.

TABLE 1

| | Day 12 | | | Day 19 | | | Day 26 | | | Day 33 | | |
|---|---|---|---|---|---|---|---|---|---|---|---|---|
| Trans- | | | root/leaf | | | root/leaf | | | root/leaf | Root | | leaf |
| formant | Root | leaf | ratio | Root | leaf | ratio | Root | leaf | ratio | root/leaf | ratio | |

| | | | | | | | | | | | | |
|---|---|---|---|---|---|---|---|---|---|---|---|---|
| ACC6H | 4.06 | 0,02 | | 4,19 | 0,09 | 47 | 1,55 | 0,05 | 31 | 2,07 | 0,19 | 11 |
| ACC6T3 | 4.39 | 0.02 | | 3.98 | 0.09 | 44 | 1.3 | 0.04 | 33 | 2.44 | 0.25 | 10 |
| 6i | 16.42 | 0.05 | | 12 | 0.25 | 48 | 4.93 | 0.12 | 41 | 8.74 | 0.57 | 15 |
| 6h | 25.87 | 1.39 | 19 | 4.94 | 0.17 | 29 | 4 | 0.27 | 15 | 4.64 | 0.32 | 15 |
| 6.1 | 32.05 | 1.01 | 32 | 22 | 0.6 | 37 | 7.9 | 0.56 | 14 | 10.3 | 0.51 | 20 |
| 6.3 | 40.61 | 1.84 | 22 | 7.75 | 0.45 | 17 | 21.68 | 0.73 | 30 | 3.97 | 0.34 | 12 |
| 6.2 | 48.87 | 1.61 | 30 | 6.49 | 0.17 | 38 | 22.01 | 1.01 | 22 | 4.55 | 0.79 | 6 |
| 6a | 6.93 | 0.14 | 50 | 1.4 | 0.01 | 140 | 1 | 0.27 | 25 | 1.48 | 0.07 | 21 |
| 6b | 8.62 | 0.73 | 12 | 5.43 | 0.78 | 7 | 2.35 | 0.26 | 9 | 2.63 | 0.19 | 14 |
| 2b | 33.02 | 4.91 | 7 | 4.93 | 1.31 | 4 | 3.69 | 1.3 | 3 | 2.8 | 0.77 | 4 |
| 6k | 107.93 | 11.57 | 9 | 32.3 | 3.09 | 10 | 11.74 | 3.65 | 3 | 6.6 | 1.75 | 4 |
| WS | −0.06 | 0.33 | | 0.1 | 0.07 | | −0.01 | 0 | | −0.04 | 0.01 | |

### BIBLIOGRAPHIC REFERENCES

F. M. AUSUBEL, R. BRENT, R. E. KINGSTON, D. D. MOORE, J. G. SEIDMAN, J. A. SMITH, K. STRUHL; 1989, Current Protocols in Molecular Biology published by Wiley Interscience.

BASTOLA, D. R., PETHE, V. V. and WINICOV, I. (1998). Alfin 1, a novel zinc finger protein in alfalfa roots that binds to promoter elements in the salt inducible MSPRP2 gène, Plant. Mol. Biol. 38, 1123-1135).

BECHTOLD N., Ellis J. PELLETIER G., 1993. Agrobacterium mediated gene transfer by infiltration of adult Arabidopsis thaliana plants. C.R. Acad. Sci. Paris 316: 1194-1199.

BOUCHEZ D., CAMILLERI C., CABOCHE M. 1993. A binary vector based on Basta resistance for in plant transformation of Arabidopsis thaliana . C.R. Acad. Sci. Paris 316: 1188-1193.

BEVAN M. 1984. Binary Agrobacterium vectors for plant transformation. Nucleic Acid Research 12: 8711-21.

BRUNAK S. ENGELBRECHT, J. and KNUDSEN, S.: Prediction of human mRNA Donor and Acceptor Sites from the DNA sequence. Journal of Molecular Biology, (1991), 220, 49-65.

BURGE, C. and KARLIN, S. (1997). Prediction of complete gene structures in human genomic DNA. J. Mol. Biol. 268, 78-94, BURGE, C. B. (1998). Modeling dependencies in pre-mRNA splicing signals. In SALZBERG, S. SEARLS, D. and KASIF; S. eds. Computational methods in molecular biology. Elsevier Science, Amsterdam, pp. 127-163.

BOUCHEZ D., VITTORIOSO P., COURTIAL B. CAMILLERI C., 1996. Kanamycin Rescue: A simple technique for the recovery of T-DNA flanking sequences. Plant Mol. Biol. Rep. 14: 115-123.

BOUCHEZ D. CAMILLERI C., CABOCHE M., 1993. A binary vector based on Basta resistance for in planta transformation of Arabidopsis thaliana . C.R. Acad. Sci. Paris 316:1188-1193.

CRAWFORD, N. M. and GLASS, A. D. M. 1998. Molecular and physiological aspects of nitrate uptake in plants. Trends in Plant Science, 3, 389-395.

CORMACK, B. P. VALDIVIA, R. H. FALKOW, S. (1996), FACS-optimized mutants of the freen fluorescent protein (GFP) Gene 173:33-38.

JEFFERSON R. A., 1987. Assaying chimeric genes in plants: the Gus gene fusion system. Plant Mol. Biol. Rep.5: 387.

HEBSGAARD S. M., P. G. KORMING, N. TOLSTRUP, J. ENGELBRECHT, P. ROUZE, S. BRUNAK: Splice site prediction in Arabidopsis thaliana DNA by combining local and global sequence information. Nucleic Acids Research, (1996), Vol.24, N° 17, 3439-3452.

HEIM U. WEBER H., BAUMLEIN H. WOBUS U. 1993. A sucrose-synthase gene of Vicia faba L. Expression pattern in developing seeds in relation to starch synthesis and metabolic regulation. Planta 191: 3494-501.

HESSE, H. SONNEWALD, K. and WILLNITZER, L. (1995). Cloning and expression analysis of sucrose-phosphate synthase from sugar beet (Beta vulgaris L.) Mol. Gen. Genet, 247, 515-520).

HWANG et al., (1995). An Arabidopsis thaliana root specific kinase homolog is induced by dehydration, ABA, and NaCl. Plant J.8: 37-43.

HWANG I., GOODMAN, H. M. (1995). An Arabidopsis thaliana root specific kinase homolog is induced by deshydration, ABA, and NaCl, The Plant Journal, 8, 37-43).

LEAH, R. TOMMERUP, H. SVENDSEN, I. and MUNDY, Y. (1991). Biochemical and molecular characterization of three barley seed proteins with antifungal properties . J. Biol. Chem. 266, 1464-1573).

MALAMY, Y. E., BENFEY, P. N. (1997). Analysis of scarecrow expression using a rapid system for assessing transgene expression in Arabidopsis roots. Plant Y. 12: 957-963.

MOLLIER P., MONTORO P., DELARUE M. BECHTOLD N. BELLINI C., PELLETIER G., 1995. Promoterless gus A expression in a large number of Arabidopsis thaliana transformants obtained by in planta infiltration method. C.R. Acd. Sci. Paris 318: 465-474.

SOUTHERN, E. M. (1975) Detection of specific sequences among DNA fragments separated by gel electrophoresis. J. Mol. Biol. 98: 503-577.

WINICOV, DEUTCH, C. E. (1994). Characterization of a CDNA clone from salt tolerant alfalfa cells that identifies salt-inducible root specific transcripts) J. Plant. Physiol. 144, 222-228).

YANG T. T. CHENG, L. KAIN, S. R. (1996) Optimized codon usage and chromophore mutations provide enhanced sensitivity with the green fluorescent protein. Nucleic Acids Res. 24: 45924593.

1. Nucleic acid comprising all or part of a plant promoter capable of directing the expression of a nucleotide sequence of interest in the cells of the root of a plant throughout the entire development of this latter, characterized in that it comprises all or part of a polynucleotide possessing at least 80% nucleotide identity with the nucleotide sequence SEQ ID No. 1 or a nucleic acid with the complementary sequence, with the exception of the sequence entered under the reference No. AC 007 289 in the EMBL data base.

2. Nucleic acid according to claim 1, characterized in that it comprises all or part of a polynucleotide hybridizing under hybridization conditions of high stringency with the nucleotide sequence SEQ ID No.1 or a nucleic acid with the complementary sequence.

3. Nucleic acid according to one of the claims 1 or 2, characterized in that it comprises one of the following sequences:
the polynucleotide extending from the nucleotide at position 1 to the nucleotide at position 2400 of the sequence SEQ ID No. 3;
the polynucleotide extending from the nucleotide at position 493 to the nucleotide at position 2400 of the sequence SEQ ID No. 3;
the polynucleotide extending from the nucleotide at position 1076 to the nucleotide at position 2400 of the sequence SEQ ID No. 3;
the polynucleotide extending from the nucleotide at position 1976 to the nucleotide at position 2400 of the sequence SEQ ID No. 3; and
the polynucleotide extending from the nucleotide at position 2040 to the nucleotide at position 2400 of the sequence SEQ ID No. 3.

4. Nucleic acid according to one of the claims 1 to 3, characterized in that it comprises a nucleotide sequence of interest placed under the control of the plant promoter.

5. Nucleic acid according to claim 4, characterized in that it is the nucleotide sequence SEQ ID No. 2.

6. Nucleic acid according to claim 4, characterized in that the nucleotide sequence of interest is selected from the coding sequences of genes interacting with parasites or pathogens, the sequences coding for the endochitinases, the sequences coding for plant proteins protecting the plant from hydric or salt stress, or also genes acting on the sugar content of the plant or on nitrate transport.

7. Nucleic acid comprising 10 to 2000 consecutive nucleotides of a nucleic acid according to one of the claims 1 to 4, useful as a nucleotide probe or primer.

8. Recombinant cloning and/or expression vector containing a nucleic acid according to one of the claims 1 to 7.

9. Recombinant vector according to claim 8, characterized in that it is selected from the vectors pBin19, 101, pBi221, pBi121 and pC-gus.

10. Recombinant vector according to one of the claims 8 or 9, characterized in that it is the vector contained in the E. coli strain deposited with the NCCM on May 25, 1999 under the access No. I-2218.

11. Recombinant cell host, characterized in that it contains a nucleic acid according to one of the claims 1 to 7 or a recombinant vector according to one of the claims 8 to 10.

12. Recombinant host cell according to claim 11, characterized in that it is of bacterial or plant origin.

13. Recombinant host cell according to claim 12, characterized in that it is an Agrobacterium tumefaciens cell.

14. Recombinant host cell according to one of the claims 11 to 13, characterized in that it is a cell of the E. coli strain deposited with the NCCM on May 25, 1999 under the access No. I-2218.

15. Recombinant plant multicellular organism, characterized in that it comprises a recombinant host cell according to one of the claims 11 to 13.

16. Transgenic plant comprising in a form integrated in its genome a nucleic acid according to one of the claims 1 to 7.

17. Transgenic plant according to claim 16, characterized in that it is colza, tobacco or maize.

18. Procedure for obtaining a transgenic plant characterized in that it comprises the following steps:
a) Production of a plant recombinant host cell according to one of the claims 11 or 12;
b) Regeneration of a whole plant from the recombinant host cell obtained in step a).
c) Selection of the plants obtained in step b) which have integrated the nucleotide sequence of interest placed under the control of the plant polynucleotide promoter.

19. Procedure for producing a transgenic plant characterized in that it comprises the following steps:
a) Production of an Agrobacterium tumefaciens recombinant host cell according to claim 13;
b) Transformation of the plant of interest by infection with the recombinant host cell obtained in step a).
c) Selection of the plants which have integrated the nucleotide sequence of interest placed under the control of the plant polynucleotide promoter.

20. Procedure for producing a transgenic plant characterized in that it comprises the following steps:
a) transfect a plant cell with a nucleic acid according to one of the claims 1 to 7 or a recombinant vector according to one of the claims 8 to 10;
b) regeneration of a whole plant from the recombinant host cells obtained in step a).
c) selection of the plants which have integrated the nucleotide sequence of interest placed under the control of the plant polynucleotide promoter.

21. Procedure for the production of a transgenic plant according to one of the claims 18 to 20, characterized in that it comprises the additional steps:
d) cross of two transgenic plants such as obtained in step c);
e) selection of the plants homozygous for the transgene.

22. Procedure for the production of a transgenic plant according to one of the claims 18 to 20, characterized in that it comprises the additional steps:
d) cross of a transgenic plant obtained in step c) with a plant of the same species;
e) selection of the plants derived from the cross of step d) which have conserved the transgene.

23. Transgenic plant such as that obtained according to the procedure in accordance with one of the claims 18 to 22.

24. Plant seed, the constituent cells of which contain in their genome a nucleic acid according to one of the claims 1 to 7.

25. Seed of a transgenic plant according to one of the claims 16, 17 and 23.


SEQUENCE LISTING (7 sequences)

SEQ ID NO: 1 - DNA, 2149 bp, Arabidopsis thaliana
gtcgaattgtgatatattgtaagcaatctgaaaagaataagtgggatatataaacaaccggcgaaagtacaagttctacctttttttggcatggaaccatgtttttaggatttactttgtaattcctgaatctttcatttcttgaattgatatttacatttttatcaaaaaaaaagtacaagttctaccaaagcacaggagttaaacaacttgtgtgtcaaatgctaatttaaagcctaatcttatgatttcccttttcttcacgatatatactgatattgatatgcacccatttgtttgtcattaacttcccactctatacatcagtatctcaaagtcgaataacaatatccataagaagtggtatattgtgaaaaaaaaaaaaaaaagtggtatactggtatatacaataccacggtctcgaattgcctcaacaatttctaggagaaaatggacgtgtctctttggttttattttattcttaataacatactctatattttaaacacttcgatgtctcgcttaaatttcgaatgtgcctaaatttctctaatcataaatcgtaaagaaaattcgtcgaagccacagggacatgcatagggcacgtagttacctttaaaaccatcaaaaatatattaatagaaaaggaaacttcctaaaagaacaatttaataaagtggataaaaaaagataagaaggtaggcagaagaaaacgtatggccgcgactcgtaacaagggacgtcccgaccactgcggagacggcgagacgctgactgattttttctttttcttttcctaaagaacgttgtttcgtgcttacaagggtcaaaaccatatccaattgttctgcctattattatataactaaagatcccctcttgtgctttgtctttattcgtgatatataatctaacttaaattagttctaaaatatatatgtcctacctatgtttctactgacctcagtccctagttagctatatggacatatgtgaaaatgacgcccaaaatttgaagagttcctcttcctgcaactaactcttatcttactcattgagctatgttaaatattgaatgttggcactctcgtattaaatatgccagttgcacctagataaaaaaacatgatagacatttagtttaaaacttgaaatgttatttgaactctttggattacgtggattgttgtatggattaaattttgaagatatttatatattgaagatgtttatatatattagagtttatatagcagaaaatattgatgtagatgttgtccttttgtagttactcttttttgttgcgtagtcctttctcctcatcctcctatgaagaaaaatccaaatagtttaaggaaatttttgtgtaattcatagtctttttcgtaaccacagttctatgtagctatcgtcatcatattcctctttgcaacaacaaaaaagatcgtttttgtaaaatttagtagggcactaaagtcgtcatttgttgtcctgtcgaaatctagcgttctgtcatccacaaataagttgtttgattcgagcttccaagattataatcttttttagatgggtcatgaagatttctaacttcgtatacgagtgtatccatataatttctaacatatacgtcttgtttttggtaggctctgcgtcttttgagaccacccccttgctaatgttttgttgcaccttagacaatccataatacgttacgtgagtcgaagttgcaccaaaatggtccaaatataatttaaatttggccacaaaacaacattttacaaacaaattcaacaaacatgcatcgtttcaaattttatttattcaatggcgttatttgttcattgtaaatattctgtttaactcactgacgaattttttaatttttcaaagaagaacatttttgatataaataacattttatggaaccaccggttaagctcgatgattttgagttttagttttgtcgttttgtgaaatcattaacgacctacatttgatccctcattactttaataattaggaatcaaacatgatgattaagttcaccaaagacgtctcttatggctattaagagtcagacgcaaggatgaccggggtcattaagacgtcttatattcaaccattactccactaattgctaattaatcag

SEQ ID NO: 2 - DNA, 4280 bp, Artificial Sequence
  Description of Artificial Sequence Construction promoter + sequence coding for the gus gene
gtcgaattgtgatatattgtaagcaatctgaaaagaataagtgggatatataaacaaccggcgaaagtacaagttctacctttttttggcatggaaccatgtttttaggatttactttgtaattcctgaatctttcatttcttgaattgatatttacatttttatcaaaaaaaaagtacaagttctaccaaagcacaggagttaaacaacttgtgtgtcaaatgctaatttaaagcctaatcttatgatttcccttttcttcacgatatatactgatattgatatgcacccatttgtttgtcattaacttcccactctatacatcagtatctcaaagtcgaataacaatatccataagaagtggtatattgtgaaaaaaaaaaaaaaaagtggtatactggtatatacaataccacggtctcgaattgcctcaacaatttctaggagaaaatggacgtgtctctttggttttattttattcttaataacatactctatattttaaacacttcgatgtctcgcttaaatttcgaatgtgcctaaatttctctaatcataaatcgtaaagaaaattcgtcgaagccacagggacatgcatagggcacgtagttacctttaaaaccatcaaaaatatattaatagaaaaggaaacttcctaaaagaacaatttaataaagtggataaaaaaagataagaaggtaggcagaagaaaacgtatggccgcgactcgtaacaagggacgtcccgaccactgcggagacggcgagacgctgactgattttttctttttcttttcctaaagaacgttgtttcgtgcttacaagggtcaaaaccatatccaattgttctgcctattattatataactaaagatcccctcttgtgctttgtctttattcgtgatatataatctaacttaaattagttctaaaatatatatgtcctacctatgtttctactgacctcagtccctagttagctatatggacatatgtgaaaatgacgcccaaaatttgaagagttcctcttcctgcaactaactcttatcttactcattgagctatgttaaatattgaatgttggcactctcgtattaaatatgccagttgcacctagataaaaaaacatgatagacatttagtttaaaacttgaaatgttatttgaactctttggattacgtggattgttgtatggattaaattttgaagatatttatatattgaagatgtttatatatattagagtttatatagcagaaaatattgatgtagatgttgtccttttgtagttactcttttttgttgcgtagtcctttctcctcatcctcctatgaagaaaaatccaaatagtttaaggaaatttttgtgtaattcatagtctttttcgtaaccacagttctatgtagctatcgtcatcatattcctctttgcaacaacaaaaaagatcgtttttgtaaaatttagtagggcactaaagtcgtcatttgttgtcctgtcgaaatctagcgttctgtcatccacaaataagttgtttgattcgagcttccaagattataatcttttttagatgggtcatgaagatttctaacttcgtatacgagtgtatccatataatttctaacatatacgtcttgtttttggtaggctctgcgtcttttgagaccacccccttgctaatgttttgttgcaccttagacaatccataatacgttacgtgagtcgaagttgcaccaaaatggtccaaatataatttaaatttggccacaaaacaacattttacaaacaaattcaacaaacatgcatcgtttcaaattttatttattcaatggcgttatttgttcattgtaaatattctgtttaactcactgacgaattttttaatttttcaaagaagaacatttttgatataaataacattttatggaaccaccggttaagctcgatgattttgagttttagttttgtcgttttgtgaaatcattaacgacctacatttgatccctcattactttaataattaggaatcaaacatgatgattaagttcaccaaagacgtctcttatggctattaagagtcagacgcaaggatgaccggggtcattaagacgtcttatattcaaccattactccactaattgctaattaatcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggtggggaattgagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaatt

SEQ ID NO: 3 - DNA, 4413 bp, Arabidopsis thaliana
aagatccacagtgaataaataataagaacggattcggtgatattgcaactatataatgaaattgaatactctgattcatcgctttgtatcaagatcgaatctctaaaaacatatactctataataaatatctgcagtcgaattgtgatatattgtaagcaatctgaaaagaataagtgggatatataaacaaccggcgaaagtacaagttctacctttttttggcatggaaccatgtttttaggatttactttgtaattcctgaatctttcatttcttgaattgatatttacatttttatcaaaaaaaaagtacaagttctaccaaagcacaggagttaaacaacttgtgtgtcaaatgctaatttaaagcctaatcttatgatttcccttttcttcacgatatatactgatattgatatgcacccatttgtttgtcattaacttcccactctatacatcagtatctcaaagtcgaataacaatatccataagaagtggtatattgtgaaaaaaaaaaaaaaaagtggtatactggtatatacaataccacggtctcgaattgcctcaacaatttctaggagaaaatggacgtgtctctttggttttattttattcttaataacatactctatattttaaacacttcgatgtctcgcttaaatttcgaatgtgcctaaatttctctaatcataaatcgtaaagaaaattcgtcgaagccacagggacatgcatagggcacgtagttacctttaaaaccatcaaaaatatattaatagaaaaggaaacttcctaaaagaacaatttaataaagtggataaaaaaagataagaaggtaggcagaagaaaacgtatggccgcgactcgtaacaagggacgtcccgaccactgcggagacggcgagacgctgactgattttttctttttcttttcctaaagaacgttgtttcgtgcttacaagggtcaaaaccatatccaattgttctgcctattattatataactaaagatcccctcttgtgctttgtctttattcgtgatatataatctaacttaaattagttctaaaatatatatgtcctacctatgtttctactgacctcagtccctagttagctatatggacatatgtgaaaatgacgcccaaaatttgaagagttcctcttcctgcaactaactcttatcttactcattgagctatgttaaatattgaatgttggcactctcgtattaaatatgccagttgcacctagataaaaaaacatgatagacatttagtttaaaacttgaaatgttatttgaactctttggattacgtggattgttgtatggattaaattttgaagatatttatatattgaagatgtttatatatattagagtttatatagcagaaaatattgatgtagatgttgtccttttgtagttactcttttttgttgcgtagtcctttctcctcatcctcctatgaagaaaaatccaaatagtttaaggaaatttttgtgtaattcatagtctttttcgtaaccacagttctatgtagctatcgtcatcatattcctctttgcaacaacaaaaaagatcgtttttgtaaaatttagtagggcactaaagtcgtcatttgttgtcctgtcgaaatctagcgttctgtcatccacaaataagttgtttgattcgagcttccaagattataatcttttttagatgggtcatgaagatttctaacttcgtatacgagtgtatccatataatttctaacatatacgtcttgtttttggtaggctctgcgtcttttgagaccacccccttgctaatgttttgttgcaccttagacaatccataatacgttacgtgagtcgaagttgcaccaaaatggtccaaatataatttaaatttggccacaaaacaacattttacaaacaaattcaacaaacatgcatcgtttcaaattttatttattcaatggcgttatttgttcattgtaaatattctgtttaactcactgacgaattttttaatttttcaaagaagaacatttttgatataaataacattttatggaaccaccggttaagctcgatgattttgagttttagttttgtcgttttgtgaaatcattaacgacctacatttgatccctcattactttaataattaggaatcaaacatgatgattaagttcaccaaagacgtctcttatggctattaagagtcagacgcaaggatgaccggggtcattaagacgtcttatattcaaccattactccactaattgctaattaatcagattaatttgtttaatacgataatgtattttgattaagtagctctcagccaacaggcaaaggataaattttggattattcaaagatttgtgggcttccaaaagcatagggaatggctccaactacattgggaaatatattatttaaatctccattcccatttgccacagtcgttggagtttattttttttttccaagtggaagaatcaattataattgtcggaatttctaaatcctacttgttggaaaacaaacccaagcaaaattagtttagaaatgtacggaaatctactatagaattatactaaacatatcaatatagctttgactttaaaattaaaacaattattgtgggcaattagttagatattttaccaggggaaataagcagcactgttcatgcactctcttcttaattactatactttgaaagaacttatatgtcactgtattgccagttgccactaatatataaacaacatttcacttgttgacatcgctgtaaatgaagtttggaacgaccctcttaacaactaatagggttaattaaccactaaatttttccaagttgtcattttgtcttaatgtgagacgtaatatctaatacgtcggtctaacctaagagttggttccgatcacaaatttttgagaagtacctttcaataaaaaatttgttgttataatttcaccgtgttaagtaaaaacttaattaggagctattttctatttgatgtgaatttgaaaatgtcttcataaaaatagtatggaaaagggaatgtaattaatatagaccacagatacaaaaagatgtcccgtgcttaacacgtctgagtcattgtcgtacccttttgccaactttttcaagtttctttcgtgaaaatgactacactttttaaaataaattgacagatgattgttgcatgcatataatattcgcaaaatgccaaattctacccttaaccaaataatgggtaatggatataaaatagttaacataaacaaaaatttggaaattttgaaaaatagagagtaaattgattttttttaaaaagtttgaattgaagtgaaaaaatatataataaaaataataaacctgtagtttgatatatatagttagatagctcaagtttgagtaacttgaagtcttgaattactttatatgtttttctcacagattatattattttcgttctatcccaagaattgggataatattctctatattcgaggcctctctcttaagaagtcggtgtataatcttaagcccttacttgacacaaggcctctattaaaaagcccaataataatttttcttttcaaagcccaacacgtcaagaggagaaagaagtgcgtttgcgtttggattgaaaacgtggcgggcggttggaaactgaatcttaaacccttcactcacattcatcttcaccgtcaaatctctgaaactgagcttcgacgatgttgggtttacgaagatctgcgacgaccttgttcgacatcagccagtctctgcttcgtaatgttacggttagtatctgtctaacacttcagcgccacatttgggttttacgattagggtttcaatagatcgaatcgattcatctctcttggtattagatttcatcaaattcaaatctatcgtcacctaaaaatctctctttcggtatgtagtatttccggggttgtttcaaagtcgtcttattgctctgtgattttggcttcttacccattttcaacagtgctatgtagaagaaagaacaaatctttgaaatcgaaaggtctaatgtatagttcaatgtctacattatgagattgccatgatattatagtcaaacgattctcctaaagcgtttacttttgtggagcattctttgttagttctatgcaaataaagttctaggaatgataattcttaaggaagcatctcaaatgttggctagttcttgtctcaggttaaaacaatgttttgcaatttgcttttagttagaattgttgacttgcttagctttttgactaactctgtctctgtgaagcaaagtttgatcaaacccattaccttatttgatttctctctttagattatacatcaatttatgtattttctttgtctacagtttcatgggttacgagtcca

SEQ ID NO: 4 - DNA, 4309 bp, Artificial Sequence
  Description of Artificial Sequence pBin19 vector insert
tctagaactagtggatcccccgggctgcagtcgaattgtgatatattgtaagcaatctgaaaagaataagtgggatatataaacaaccggcgaaagtacaagttctacctttttttggcatggaaccatgtttttaggatttactttgtaattcctgaatctttcatttcttgaattgatatttacatttttatcaaaaaaaaagtacaagttctaccaaagcacaggagttaaacaacttgtgtgtcaaatgctaatttaaagcctaatcttatgatttcccttttcttcacgatatatactgatattgatatgcacccatttgtttgtcattaacttcccactctatacatcagtatctcaaagtcgaataacaatatccataagaagtggtatattgtgaaaaaaaaaaaaaaaagtggtatactggtatatacaataccacggtctcgaattgcctcaacaatttctaggagaaaatggacgtgtctctttggttttattttattcttaataacatactctatattttaaacacttcgatgtctcgcttaaatttcgaatgtgcctaaatttctctaatcataaatcgtaaagaaaattcgtcgaagccacagggacatgcatagggcacgtagttacctttaaaaccatcaaaaatatattaatagaaaaggaaacttcctaaaagaacaatttaataaagtggataaaaaaagataagaaggtaggcagaagaaaacgtatggccgcgactcgtaacaagggacgtcccgaccactgcggagacggcgagacgctgactgattttttctttttcttttcctaaagaacgttgtttcgtgcttacaagggtcaaaaccatatccaattgttctgcctattattatataactaaagatcccctcttgtgctttgtctttattcgtgatatataatctaacttaaattagttctaaaatatatatgtcctacctatgtttctactgacctcagtccctagttagctatatggacatatgtgaaaatgacgcccaaaatttgaagagttcctcttcctgcaactaactcttatcttactcattgagctatgttaaatattgaatgttggcactctcgtattaaatatgccagttgcacctagataaaaaaacatgatagacatttagtttaaaacttgaaatgttatttgaactctttggattacgtggattgttgtatggattaaattttgaagatatttatatattgaagatgtttatatatattagagtttatatagcagaaaatattgatgtagatgttgtccttttgtagttactcttttttgttgcgtagtcctttctcctcatcctcctatgaagaaaaatccaaatagtttaaggaaatttttgtgtaattcatagtctttttcgtaaccacagttctatgtagctatcgtcatcatattcctctttgcaacaacaaaaaagatcgtttttgtaaaatttagtagggcactaaagtcgtcatttgttgtcctgtcgaaatctagcgttctgtcatccacaaataagttgtttgattcgagcttccaagattataatcttttttagatgggtcatgaagatttctaacttcgtatacgagtgtatccatataatttctaacatatacgtcttgtttttggtaggctctgcgtcttttgagaccacccccttgctaatgttttgttgcaccttagacaatccataatacgttacgtgagtcgaagttgcaccaaaatggtccaaatataatttaaatttggccacaaaacaacattttacaaacaaattcaacaaacatgcatcgtttcaaattttatttattcaatggcgttatttgttcattgtaaatattctgtttaactcactgacgaattttttaatttttcaaagaagaacatttttgatataaataacattttatggaaccaccggttaagctcgatgattttgagttttagttttgtcgttttgtgaaatcattaacgacctacatttgatccctcattactttaataattaggaatcaaacatgatgattaagttcaccaaagacgtctcttatggctattaagagtcagacgcaaggatgaccggggtcattaagacgtcttatattcaaccattactccactaattgctaattaatcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggtggggaattgagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaatt

SEQ ID NO: 5 - DNA, 7599 bp, Artificial Sequence
  Description of Artificial Sequence T-DNA of pGKB5
ggaaacagctatgaccatgattacgccaagctcggaattaaccctcactaaagggaacaaaagctggagctccaccgcggtggcggccgctctagaggatccccccacagacagctccgtagccctcgttctccttggagttcttcgggaaatggatctttcgattcccgatgatgtctctcttatctgctttgacgacgccgactggacatccgctataacgccgccattgaccgtgatttcgcaacctgtcagggatctcgcgacggctgccacagaagacctgatcgcccgcttaaagggcgagacttcagccccacccaaggaaactcttctcccggcggttctcatagagcgcggttccgtaagcggttcttcgcaaggtcggggttgcataccgaactcgcgaaacgtcggcgactgagctcccgaggcgcgttgacaagatgccacgaagggaatggaagacagccgatattgcaattgtcttcgtggactgctttcgggacgtaaggcgcaagccatcatcaccgccgtcctaaacaaacatacctccacacaaatttatctacctgaccacaagatatatcctgtcacacgatttattaaacgctgcacttggggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggtggggaattgagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattcgatcgaggggatcgagcccctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgaacgttattagttcgccgctcggtgtgtcgtagatactagcccctggggccttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccacatgatcatgggccggatctttgattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaatcgttgcggttctgtcagttccaaacgtaaaacggcttgtcccgcgtcatcggcgggggtcataacgtgactcccttaattctccgctcatgatcctgtttcctgtgtgaaattgttatccgctcacaattccacacattatacgagccggaagcataaagtgtaaagcctggggtgcctaatgagtgagctaactcacattaattgcgttgcgctcactgcccgctttccagtcgggaaacctgtcgtgccagctgcattaatgaatcggaattgacggatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatcggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatctcacgcgtctaggatccgatggatcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaacatctacaaattgccttttcttatcgaccatgtacatcaagcttatcgataccgtcggctattggtaataggacactgggattcgtcttggacaactttccttctcatctaagcgtagacaaccctcaactggaaacgggccggactccagggcgtgtgccaggtgcccacggaatagttttggccagacccttgaaaatccgattcagtacaatcgattgccctcatttttacgttggcatatatcctgccaaacagccaacaacgcgcgtgcggtgaataggaaagcgtttgagttgcttgctcatatcgtgacggttgacagcacaggttgaccgcttgatgattcgtacgagccgccaaacattggctgtcgtaatgatataccatgtcagaacagcaatccgatggggcggaaagcattatcttaatgcacacggaaatggcgcgtcggtgggtggaatacaccgacatagaggccgtaagttctgcatggtcatcgtcggaaaggtggcagcaggcgcacggctgtggcctcttgctctttcagcgtgaaatgcgtgttgaaagaataatcgaagagagcgtccgctcgacaccttcaattatgccgatttgatcgatgaactgatcgagctctgaaatcgaaggggcttcgataatcgcaatcaaatcaaaagtgccactcacagaatgaagagcgataacggccgtgaccttcccaagggaggccgtcacctgtgaaagcgccttcgtaatggtgatcagaatatgggctcgaaccaagctcgagacctcgagggggggcccggtacccaattcgccctatagtgagtcgtattacaattcactggccgtcgttttac

SEQ ID NO: 6 - DNA, 31 bp, Arabidopsis thaliana
ggcaagcttgtaatacgactcactatagggc

SEQ ID NO: 7 - DNA, 25 bp, Arabidopsis thaliana
ctagggatccagccattccctatgc